(19) United States
(12) Patent Application Publication
(10) Pub. No.: US 2019/0054454 A1
(43) Pub. Date: Feb. 21, 2019

(54) MECHANICALLY STRONG CATALYST AND CATALYST CARRIER, ITS PREPARATION, AND ITS USE

(71) Applicant: SHELL OIL COMPANY, Houston, TX (US)

(72) Inventor: Hendrik Albertus COLIJN, Amsterdam (NL)

(21) Appl. No.: 15/770,691

(22) PCT Filed: Oct. 25, 2016
(86) PCT No.: PCT/EP2016/075591

§ 371 Date: Apr. 24, 2018

(30) Foreign Application Priority Data
Oct. 26, 2015 (EP) 15191399.3

(51) Int. Cl. B01J 27/057; B01J 21/08; B01J 29/40; B01J 35/00; B01J 37/00; B01J 37/04; B01J 37/08; C07C 5/48; C07C 51/25

(52) U.S. Cl. CPC B01J 27/0576; C07C 2529/40; B01J 29/40; B01J 35/0013; B01J 37/0018; B01J 37/04; B01J 37/08; C07C 5/48; C07C 51/25; C07C 51/252; C07C 2523/20; C07C 2523/22; C07C 2523/28; C07C 2527/057; C07C 2521/08; B01J 21/08

(57) ABSTRACT

The invention concerns catalyst or a catalyst carrier comprising 35 to 99.9 wt % of metal oxide and 0.1 to 50 wt % of silanized silica particles, calculated on the total weight of the catalyst or catalyst carrier. The invention further relates to a process to prepare the catalyst or catalyst carrier. The invention also relates to the use of the catalyst, or a catalyst comprising the catalyst carrier, in a catalytic reaction.

### FIELD OF THE INVENTION

The present invention relates to a process for preparing a strong catalyst or catalyst carrier. The invention further relates to a process to prepare the catalyst or catalyst carrier. The invention also relates to the use of the catalyst, or a catalyst comprising the catalyst carrier, in a catalytic reaction.

### BACKGROUND OF THE INVENTION

Catalysts used in catalytic reactions are often subjected to severe conditions, and thus need to have a sufficient strength.

Catalysts may comprise or may not comprise a carrier. If a catalyst comprises a carrier which is not catalytically active, it also contains a catalytically active material. Catalytically active material may be supplied to a carrier by any suitable method, such as impregnation. An alternative method is extrusion.

A catalyst comprising a carrier preferably comprises a carrier having sufficient strength.

During a catalytic reaction a catalyst may be subjected to high temperatures and/or to high pressures. Additionally or alternatively, a catalyst may be subjected to mechanical stress before and/or during a catalytic reaction.

A catalyst may be subjected to, for example, dynamic stress, static stress, compression stress, shear stress, impact stress, abrasion, friction, and/or collision. One example is catalyst particles colliding with each other and/or with the walls or internals of a reactor in a fluidized bed reactor. Another example is the weight of the catalyst bed on catalyst particles at the bottom of a fixed bed of catalyst particles. Further examples are impact stresses during transport, handling and storage operations of a catalyst or catalyst carrier.

The aim of the present invention is to provide a strong catalyst or catalyst carrier, a method to prepare the catalyst or catalyst carrier, and the use thereof, wherein said catalyst or a catalyst comprising said catalyst carrier can be suitably used in a catalytic reaction, especially in an alkane oxidative dehydrogenation (alkane ODH) and/or alkene oxidation reaction.

### SUMMARY OF THE INVENTION

The present invention relates to a catalyst or a catalyst carrier comprising:
35 to 99.9 wt %, preferably 45 to 99.9 wt %, more preferably 75 to 99.9 wt %, of metal oxide and
0.1 to 50 wt %, preferably 0.1 to 20 wt %, of silanized silica particles,
   calculated on the total weight of the catalyst or catalyst carrier.

The amount of metal oxide indicated above does not include the amount of silanized silica particles. With the amount of metal oxide is meant metal oxide(s) present in addition to the silanized silica particles.

Further, the present invention relates to a process for the preparation of a catalyst or catalyst carrier, comprising the steps of:

(a) contacting
metal oxide,
silanized silica, suitably silanized silica particles, more suitably a dispersion of silanized silica particles, even more suitably a dispersion of silanized colloidal silica particles,
a solvent and/or one or more shaping aids;
   (b) shaping or forming, suitably shaping, the material obtained in step (a);
   (c) drying and/or heating, suitably heating, the material obtained in step (b):
at a temperature in the range of from 60 to 700° C., preferably 60 to 450° C.,
preferably in air.

Still further, the present invention relates to use of the above-described catalyst, a catalyst prepared by the above-described process, a catalyst comprising the above-described catalyst carrier or a catalyst comprising a catalyst carrier prepared by the above-described process in a catalytic reaction.

Yet still further, the present invention relates to a process of the oxidative dehydrogenation of an alkane containing 2 to 6 carbon atoms and/or the oxidation of an alkene containing 2 to 6 carbon atoms, comprising contacting oxygen and the alkane containing 2 to 6 carbon atoms and/or the alkene containing 2 to 6 carbon atoms with the above-described catalyst, a catalyst prepared by the above-described process, a catalyst comprising the above-described catalyst carrier or a catalyst comprising a catalyst carrier prepared by the above-described process.

### BRIEF DESCRIPTION OF THE DRAWING

FIG. 1 shows performance data of a catalyst according to the invention prepared by a process according to the invention in converting ethane into ethylene by oxidative dehydrogenation of ethane (ethane ODH).

### DETAILED DESCRIPTION OF THE INVENTION

In the present invention, the catalyst or catalyst carrier may be a particulate catalyst or particulate carrier. It may be a heterogeneous catalyst or a heterogeneous carrier in the form of particles. The particles may be of any size suitable to be used in a reactor.

The particles may be small enough to be used in a slurry bed reactor such as a three-phase slurry bubble column. The particles may be small enough to be used in a fluidized bed reactor, such as an entrained fluidized bed reactor or a fixed fluidized bed reactor. The particles may be of sufficiently small size to be used in an ebulated bed reactor.

The particles may be large enough to be arranged in a catalyst bed in a reactor. In that case the reactor may be a (multi-) tubular fixed bed reactor. Such a catalyst bed may comprise pellets, extrudates, or catalyst on a metal support (like a metal wire or metal flake), preferably extrudates.

The invention has been found to be very advantageous. One advantage of the catalyst or catalyst carrier according to the invention is that it is strong. Even a catalyst or catalyst carrier comprising zeolite or a metal oxide which is obtainable in powder form, such as metal oxides comprising molybdenum, is strong.

One advantage of the process of the present invention is that it is suitable to prepare a strong catalyst or catalyst carrier.

Another advantage of the process of the invention is that it is suitable to prepare strong catalyst or catalyst carriers from powders, for example from metal oxide powder or from zeolite powder. It is very advantageous that strong shapes, especially extrudates, can be prepared from metal oxides which are obtainable in powder form, such as metal oxides comprising molybdenum, and from zeolite powder.

Catalysts and catalyst carriers according to the invention, or prepared according to the process of the invention, especially show a high Flat Plate Crushing Strength and/or a high attrition resistance.

Further, the catalyst according to the invention or a catalyst prepared by the process according to the invention can advantageously be used in catalytic reactions, such as in alkane oxidative dehydrogenation (ODH) and/or alkene oxidation, suitably in converting ethane into ethylene by oxidative dehydrogenation (ethane ODH).

Flat Plate Crushing Strength

Flat plate crushing strength is generally regarded as a test method to measure strength (in N/cm) at which catalyst particles collapse. The strength can be related to the compressive strength of concrete being tested in a similar test method (i.e. 10 cm cubed sample between plates), but then on a larger scale.

Currently, there is no national or international standard test or ASTM for flat plate crushing strength. However, the “compression test” for concrete, used to measure compressive strength, is well known in the art. Furthermore the general shapes of catalysts or catalyst carriers, for example the shape of spray dried particles, and extrudates such as cylinders or trilobes, are well known. The flat plate crushing test strength is independent of product quality in terms of performance in a catalytic reaction.

Naturally, any comparison of flat plate crushing strength must be made between equivalently shaped particles. Usually, it is made between the “top” and “bottom” sides of particles. Where the particles are regularly shaped such as squares, it is relatively easy to conduct the strength tests and make direct comparison. It is known in the art how to make comparisons where the shapes are not so regular, e.g. by using flat plate crushing strength tests.

Attrition Resistance

The attrition index is a measure for the resistance to attrition.

Attrition Index of Small Catalyst Particles

The attrition index of particles that are small enough to be used in a fluidized bed reactor, slurry bed reactor, or ebulated bed reactor, can be determined as follows.

The test is performed on a slurry of the catalyst particles, e.g. a thin mixture of the solid catalyst particles in a liquid.

The two parameters that are used to define resistance against attrition are Average Particle Diameter (APD) and fr<10. APD is measured as the volume weighted average particle diameter, D(4,3), or the De Broucker mean. Fr<10 is the volume fraction of particles having a diameter of <10 μm.

The attrition as used herein is defined as the percent decrease in APD during a test. In addition the attrition rate is further defined as the absolute increase in the amount of particles having a diameter of less than 10 μm, the ‘fr<10’. The latter parameter gives additional and important information on the amount of so-called “fines” that may be formed during a test. Fines are detrimental to process operations in slurry as they may clog the filters which are used for catalyst/product separation in slurry operation.

The APD is defined as:

Δ(APD)=APDₜ₌₀−APDₜ₌₃₀*100(%)

APDₜ₌₀

The increase in fr<10 is defined as

Δ(fr<10)=[fr<10]ₜ₌₃₀−[fr<10]ₜ₌₀

In order to determine the repeatability of the test a series of tests needs to be carried out. Repeatability is defined as: a value below which the absolute difference between two test results obtained with the same method on identical test material under the same conditions may be expected to lie with a specified probability. In the absence of other information, the confidence level is 95%. The relative standard deviations, for both parameters, are less than 5%.

The test also needs to be reliable over longer periods of time, i.e. the equipment should not show any signs of wearing down and attrition rate should remain constant. In order to verify that this is the case, a reference catalyst may be tested regularly, for example each (series of) test(s) may be preceded by a reference test.

Catalysts may be tested at a low volume concentration. For example, catalyst particles may be tested at 5% v/v concentration, i.e. the volume-based concentration, which is calculated using the following equation:

$$\% v \text{/} v = \frac{Mcat}{Mcat [ 1 - PV * PAD ] + [ Ml \text{/} dL ] * PAD} * 100$$

Where Mcat is the mass of catalyst, ML is the mass of the liquid, dL is the density of the liquid, PV is the pore volume of the catalyst (in ml/g, e.g. measured manually by adding small amounts of water to a known mass of catalyst until wetness occurs), and PAD is the particle density of the catalyst, calculated from PV and the skeletal density, SKD, of the catalyst:

$$PAD = \frac{1}{( 1 \text{/} SKD ) + PV} ( g \text{/} ml )$$

$$SKD = Σ MFi * di ( g \text{/} ml )$$

The above test is reliable, simple, quick and efficient, being conveniently performed in water as the liquid medium at a temperature of 20° C. The test mimics the shear conditions occurring in a commercial fluidized bed catalytic process (such as in a pump loop, due to stirrers, or due to other internals) by exposing the catalyst particles to a high shear mixer/disperser for a specified period of time. The change in the particle size distribution of the catalyst is a measure of its strength or its attrition resistance. The test can be conducted with an estimated repeatability of better than ±5%.

Attrition Index of Larger Catalyst Particles

The attrition index of larger catalyst particles, especially particles that are large enough to be used in an ebulated bed reactor, or to be arranged in a catalyst bed, for example catalyst particles suitable to be used in a (multi-)tubular fixed bed reactor, can be determined as follows.

The catalyst particles may be rotated within a (simple) drum with one internal baffle plate, over a standard number of drum rotations. The loss of material can then be determined as the change in weight of material below a certain size, e.g. below 0.84 mm, judged as being “fines”. Fines are detrimental to process operations as they may clog any filters used. Fines can, for example, also create a large pressure build up in long tubular reactors.

Details on the Catalyst and Catalyst Carrier

The invention relates to a catalyst or a catalyst carrier comprising:
35 to 99.9 wt %, preferably 45 to 99.9 wt %, more preferably 75 to 99.9 wt %, of metal oxide and
0.1 to 50 wt % of silanized silica particles,
   calculated on the total weight of the catalyst or catalyst carrier.

The amount of metal oxide indicated above does not include the amount of silanized silica particles. With the amount of metal oxide is meant metal oxide(s) present in addition to the silanized silica particles.

In the context of the present invention, in a case where a stream or catalyst or catalyst carrier comprises two or more components, these components are to be selected in an overall amount not to exceed 100 vol. % or 100 wt. %.

In particular, the invention relates to a catalyst or a catalyst carrier comprising:
35 to 99.9 wt %, preferably 45 to 99.9 wt %, more preferably 75 to 99.9 wt %, of metal oxide and
0.1 to 50 wt % of silanized colloidal silica particles,
   calculated on the total weight of the catalyst or catalyst carrier.

In the present specification, by “silanized colloidal silica particles” reference is made to silanized silica particles that may be used in a process for the preparation of the catalyst or the catalyst carrier of the present invention, in which process a dispersion of silanized silica particles may be used.

Further, in the present specification, by “silanized silica” particles reference is made to particles comprising silanized silica. Still further, in the present specification, by “silanized silica” reference is made to silanized silica that has been prepared by reacting silica with a silane. Said silane may be of formula Si(X)₄, wherein X may be the same or different and may be selected from the group of halogen, alkyl and alkoxy. Said halogen may be fluorine (F), chlorine (Cl), bromine (Br) or iodine (I). Said alkyl may comprise 1 to 10 carbon atoms, suitably 1 to 5 carbon atoms. Said alkoxy may comprise 1 to 10 carbon atoms, suitably 1 to 4 carbon atoms. Further, said alkyl group may be substituted, preferably at its terminal position, preferably by a hydrophilic group. Said hydrophilic group may comprise heteroatoms, preferably one or more oxygen atoms. Said hydrophilic group may comprise one or more moieties selected from the group consisting of ether and hydroxyl moieties. An example of an ether moiety is an epoxy moiety. Preferably, said hydrophilic group comprises at least one ether moiety and at least one hydroxyl moiety. A suitable hydrophilic group is a glycidoxy group (2,3-epoxy-1-propoxy group) or the equivalent thereof wherein the epoxy ring has been opened into a diol, that is to say a 2,3-dihydroxy-1-propoxy group. A suitable silane of formula Si(X)₄ is a silane wherein one substituent is an alkyl group which is substituted, preferably at its terminal position, preferably by a hydrophilic group as described above, and wherein the other three substituents are halogen and/or alkoxy as described above, preferably alkoxy. A suitable hydrophilic substituted alkyl group is (3-glycidoxy)propyl or the equivalent thereof wherein the epoxy ring has been opened into a diol, that is to say a (2,3-dihydroxy-1-propoxy)propyl.

Preferably, in the present invention, the catalyst or catalyst carrier is an extrudate. In the present specification, an “extrudate” means a product of an “extrusion” process which is a process used to create objects of a fixed cross-sectional profile, wherein a material is pushed through a die of the desired cross-section.

Catalyst and Catalyst Carrier

A catalyst of the present invention may be catalytically active, or it may become catalytically active after activation.

Some catalysts are active when fresh prepared or after regeneration. Other catalysts may need to be subjected to an activation step or procedure to make them catalytically active. Activation of a fresh prepared or a regenerated catalyst may be carried out in any known manner and under conventional conditions.

For example, some catalysts may be activated by subjecting it to a heat treatment. As another example, some catalyst may be activated by reducing catalytically active metal present in the catalyst. Reduction may, for example, be performed by contacting the catalyst with hydrogen or a hydrogen-containing gas, for instance at elevated temperatures.

A catalyst carrier of the present invention may be used as carrier when preparing a catalyst. Catalytically active material, or a material that becomes catalytically active after activation, may be applied to the carrier. Examples of suitable application methods are adsorption, vapour deposition, spray drying, coating and impregnation.

Metal Oxide

The catalyst or catalyst carrier of the invention comprises 35 to 99.9 wt %, preferably 45 to 99.9 wt %, more preferably 75 to 99.9 wt %, of metal oxide, calculated on the total weight of the catalyst or catalyst carrier.

The metal oxide in the catalyst or catalyst carrier preferably comprises one or more of the following: antimony oxide, tungsten oxide, nickel oxide, niobium oxide, bismuth oxide, tin oxide, copper oxide, chromium oxide, cobalt oxide, barium oxide, manganese oxide, magnesium oxide, lanthanum oxide, cerium oxide, alumina, zirconia, ruthenia, iron oxide, molybdenum oxide, molybdenum-vanadium oxide, molybdenum-vanadium-niobium oxide, molybdenum-vanadium-niobium-tellurium oxide, molybdenum-vanadium-niobium-tellurium-antimony oxide, molybdenum-vanadium-niobium-antimony oxide, molybdenum-vanadium-antimony oxide, titania, silica, silica alumina, and zeolite.

The catalyst or catalyst carrier may, for example, comprise titania and cobalt oxide. Or it may, for example, comprise silica and iron oxide.

The catalyst or catalyst carrier may, for example, comprise zeolite.

The catalyst or catalyst carrier may, for example, comprise a mixed metal oxide comprising molybdenum, or a mixed metal oxide comprising molybdenum and vanadium, or a mixed metal oxide comprising molybdenum, vanadium and niobium.

Suitable preparation methods for such mixed metal oxides are known to a person skilled in the art. Suitable preparation methods are, for example, described in WO2015082598, U.S. Pat. No. 5,534,650, Manuel Baca et al., Applied Catalysis A: General 279, pages 67-77, 2005; W. D. Pyrz et al., PNAS, vol 107, no. 14, April 2010 and the Supporting Information: Pyrz et al. 10.1073/pnas. 1001239107; E. K. Novakova et al., Journal of Catalysis 211, pages 226-234, 2002.

The metal oxide in the catalyst or catalyst carrier more preferably comprises one or more of the following: molybdenum oxide, molybdenum-vanadium oxide, molybdenum-vanadium-niobium oxide, molybdenum-vanadium-niobium-tellurium oxide, molybdenum-vanadium-niobium-tellurium-antimony oxide, molybdenum-vanadium-niobium-antimony oxide, molybdenum-vanadium-antimony oxide, titania, silica, cerium oxide, silica alumina and zeolite.

The catalyst or catalyst carrier may, for example, comprise a mixed metal oxide comprising:
molybdenum, vanadium and antimony, or
molybdenum, vanadium, niobium and optionally tellurium or antimony.

Depending on the use, the catalyst or catalyst carrier suitably comprises a mixed metal oxide comprising Mo/V/Sb, Mo/V/Nb, Mo/V/Nb/Sb, or Mo/V/Nb/Te in the orthorhombic M1 crystalline phase and/or in the pseudo-hexagonal M2 crystalline phase.

In a suitable preparation method for M1 mixed metal oxides comprising Mo/V/Sb, Mo/V/Nb, Mo/V/Nb/Sb, or Mo/V/Nb/Te, a solution or a slurry comprising the metals may be prepared. Preferably an aqueous solution or an aqueous slurry comprising the metals is prepared. The solution or slurry may be prepared using metal salts and/or metal acids such as ammonium heptamolybdate, vanadate, vanadyl sulfate, ammonium metavanadate, telluric acid, antimony tri-oxide, and ammonium niobate oxalate. Optionally organic acids or anorganic acids such as oxalic acid and/or nitric acid are added to the (aqueous) solution or slurry to reduce the pH. Upon drying solids are obtained. The solids may be subjected to a heat treatment in air. In a preferred embodiment the solids are subjected to a heat treatment in air, followed by heating in an inert atmosphere, e.g. under nitrogen. Optionally, after such heat treatment, the solids are washed, for example with water. In a preferred preparation method for M1 mixed metal oxides comprising Mo/V/Nb, Mo/V/Nb/Sb, or Mo/V/Nb/Te, an (aqueous) solution or slurry comprising the metals is prepared and dried, the solids are optionally milled to a fine powder, and then the solids are calcined in air, e.g. static air, at a temperature of about 300° C. for about 1 to 10 hours, and then heated under nitrogen, e.g. a nitrogen stream, at about 600° C. for about 0.5 to 5 hours. In a preferred preparation method for M1 mixed metal oxides comprising Mo/V/Sb, an (aqueous) solution or slurry comprising the metals is prepared and dried in an autoclave.

In case during the preparation both M1 crystalline phase and M2 crystalline phase are formed, the M2 preferably is partially or completely removed. Removal of M2 from M1 crystalline mixed metal oxide may, for example be performed by washing M2 crystalline material out by means of oxalic acid, hydrogen peroxide, nitric acid, citric acid, and/or methanol.

In the present invention, the metal oxide may be a mixed metal oxide of molybdenum, vanadium, niobium and optionally tellurium which may have the following formula:

Mo₁VₐTe_{b}Nb_{c}Oₙ

wherein:

a, b, c and n represent the ratio of the molar amount of the element in question to the molar amount of molybdenum (Mo);

a (for V) is from 0.01 to 1, preferably 0.05 to 0.60, more preferably 0.10 to 0.40, more preferably 0.20 to 0.35, most preferably 0.25 to 0.30;

b (for Te) is 0 or from >0 to 1, preferably 0.01 to 0.40, more preferably 0.05 to 0.30, more preferably 0.05 to 0.20, most preferably 0.09 to 0.15;

c (for Nb) is from >0 to 1, preferably 0.01 to 0.40, more preferably 0.05 to 0.30, more preferably 0.10 to 0.25, most preferably 0.14 to 0.20; and

n (for 0) is a number which is determined by the valency and frequency of elements other than oxygen.

The above-mentioned mixed metal oxide of molybdenum, vanadium, niobium and optionally tellurium may be prepared in many ways. Examples of catalysts comprising such mixed metal oxide and processes for preparing these, are for example disclosed in above-mentioned U.S. Pat. No. 7,091,377, WO2003064035, US20040147393, WO2010096909 and US20100256432, the disclosures of which are herein incorporated by reference.

The catalyst or catalyst carrier may, for example, comprise zeolite. Optionally, the catalyst or catalyst carrier comprises zeolite and a metal. It may, for example, comprise zeolite and platinum (Pt) and/or tin (Sn).

Silanized Silica Particles

The catalyst or catalyst carrier of the invention comprises 0.1 to 50 wt % of silanized silica particles, calculated on the total weight of the catalyst or catalyst carrier.

Silanized silica particles preferably are particles as described in WO2004035474, or WO2010103020, or WO 2012130763.

Suitable silanized colloidal silica particles are obtainable from AkzoNobel, for example silanized Bindzil CC, including Bindzil CC301 and Bindzil CC151 HS.

Preferably the catalyst or catalyst carrier of the invention comprises silanized silica particles having an average particle diameter of 1 to 1,000 nm, preferably 2 to 100 nm, more preferably 2 to 40 nm, most preferably 2 to 10 nm. In the present specification, by “average particle diameter” reference is made to a volume-based average particle diameter. Suitably, the average particle diameter is determined by a method for measuring the particle diameter distribution and then calculating the average particle diameter. An example of such method is laser diffraction (Dynamic Light Scattering). A suitable dynamic light scattering system for measuring particle diameters in the range of from 0.3 nanometers (nm) to 10.0 micrometers (microns) is “Zetasizer Nano S” available from Malvern.

Preferably the catalyst or catalyst carrier of the invention comprises 0.1 to 25 wt %, preferably 0.1 to 10 wt %, more preferably 0.1 to 7 wt % of silanized silica particles, calculated on the total weight of the catalyst or catalyst carrier.

In the present invention, the silanized silica may be as described above. In particular, the catalyst or catalyst carrier of the invention may comprise silanized silica particles silanized with epoxy silane, more preferably silanized with epoxy silane with a glycidoxy group and/or a glycidoxypropyl group, even more preferably silanized with epoxy silane with a glycidoxy group and/or a glycidoxypropyl group chosen from the group of gamma-glycidoxypropyl trimethoxysilane, gamma-glycidoxypropyl methyldiethoxysilane, and (3-glycidoxypropyl)triethoxy silane.

Silica and/or Cerium Oxide and One or More Further Metal Oxides

As discussed above, the invention relates to a catalyst or a catalyst carrier comprising:
35 to 99.9 wt %, preferably 45 to 99.9 wt %, more preferably 75 to 99.9 wt %, of metal oxide and
0.1 to 50 wt % of silanized silica particles,
   calculated on the total weight of the catalyst or catalyst carrier.

As mentioned above, the amount of metal oxide indicated above does not include the amount of silanized silica particles. With the amount of metal oxide is meant metal oxide(s) present in addition to the silanized silica particles.

In a preferred embodiment the catalyst or catalyst carrier comprises silanized silica particles, silica and/or cerium oxide, and one or more further metal oxides.

Silica, especially silicon dioxide powder, for example precipitated or fumed silica powder, preferably precipitated silica powder, can be obtained from several companies, for example Sigma Aldrich and Evonik.

In a preferred embodiment the catalyst comprises mixed metal oxide, silanized silica particles, and silica and/or cerium oxide. In that case the catalyst preferably comprises 1 to 30 wt % of silica and/or cerium oxide, calculated on the total weight of the catalyst. More preferably the total amount of silanized silica particles and silica and/or cerium oxide in the catalyst is in that case 2 to 40 wt %, even more preferably 2 to 25 wt %, calculated on the total weight of the catalyst.

In a more preferred embodiment the catalyst or catalyst carrier comprises:
in the range of from 0.1 to 50 wt % of silanized silica particles, and
silica and/or cerium oxide, and
one or more of the following:
   molybdenum oxide, molybdenum-vanadium oxide, molybdenum-vanadium-niobium oxide, molybdenum-vanadium-niobium-tellurium oxide, molybdenum-vanadium-niobium-tellurium-antimony oxide, molybdenum-vanadium-antimony oxide, titania, silica alumina and zeolite;
      whereby the catalyst or catalyst carrier comprises in total 35 to 99.9 wt %, preferably 45 to 99.9 wt %, more preferably 75 to 99.9 wt %, of metal oxide.

Even more preferably the catalyst or catalyst carrier comprises:
in the range of from 0.1 to 50 wt % of silanized silica particles, and
in the range of from 1 to 30 wt % of silica and/or cerium oxide, calculated on the total weight of the catalyst or catalyst carrier, and
one or more of the following:
   molybdenum oxide, molybdenum-vanadium oxide, molybdenum-vanadium-niobium oxide, molybdenum-vanadium-niobium-tellurium oxide, molybdenum-vanadium-niobium-tellurium-antimony oxide, molybdenum-vanadium-antimony oxide, titania, silica alumina and zeolite,
      whereby the catalyst or catalyst carrier comprises in total 35 to 99.9 wt %, preferably 45 to 99.9 wt %, more preferably 75 to 99.9 wt %, of metal oxide.

Still more preferably the catalyst or catalyst carrier comprises:
in the range of from 0.1 to 50 wt % of silanized silica particles, and
in the range of 1 to 30 wt % of silica and/or cerium oxide, calculated on the total weight of the catalyst or catalyst carrier, and
one or more of the following:
   molybdenum oxide, molybdenum-vanadium oxide, molybdenum-vanadium-niobium oxide, molybdenum-vanadium-niobium-tellurium oxide, molybdenum-vanadium-niobium-tellurium-antimony oxide, molybdenum-vanadium-antimony oxide, titania, silica alumina and zeolite.
      whereby the catalyst or catalyst carrier comprises in total 35 to 99.9 wt %, preferably 45 to 99.9 wt %, more preferably 75 to 99.9 wt %, of metal oxide, and whereby the total amount of silanized silica particles and silica and/or cerium oxide in the catalyst is 2 to 40 wt %, preferably 2 to 25 wt %, calculated on the total weight of the catalyst or catalyst carrier.

Process for Preparing Catalyst or Catalyst Carrier

The present invention also relates to a process for the preparation of a catalyst or catalyst carrier, comprising the steps of:

(a) contacting
metal oxide,
silanized silica, suitably silanized silica particles, more suitably a dispersion of silanized silica particles, even more suitably a dispersion of silanized colloidal silica particles,
a solvent and/or one or more shaping aids;
   (b) shaping or forming, suitably shaping, the material obtained in step (a);
   (c) drying and/or heating, suitably heating, the material obtained in step (b):
at a temperature in the range of from 60 to 700° C., preferably 60 to 450° C.,
preferably in air.

In the present specification, the phrase “dispersion of silanized colloidal silica particles” has the same meaning as the phrase “dispersion of colloidal silanized silica particles” and vice versa.

The above-described features, preferences and embodiments for the metal oxide, silanized silica and silanized silica particles in the catalyst or catalyst carrier according to the invention also apply to the metal oxide, silanized silica and silanized silica particles that may be used in step (a) of the above-mentioned process according to the invention for preparing a catalyst or catalyst carrier.

Preferably, in the process of the present invention, a catalyst or a catalyst carrier as described above is prepared.

A catalyst prepared according to the process of the present invention may be catalytically active, or it may become catalytically active after activation.

Some catalysts are active when fresh prepared or after regeneration. Other catalysts may need to be subjected to an activation step or procedure to make them catalytically active. Activation of a fresh prepared or a regenerated catalyst may be carried out in any known manner and under conventional conditions.

For example, some catalysts may be activated by subjecting it to a heat treatment. As another example, some catalyst may be activated by reducing catalytically active metal present in the catalyst. Reduction may, for example, be performed by contacting the catalyst with hydrogen or a hydrogen-containing gas, for instance at elevated temperatures.

A catalyst carrier prepared according to the process of the present invention may be used as carrier when preparing a catalyst. Catalytically active material, or a material that becomes catalytically active after activation, may be applied to the carrier. Examples of suitable application methods are adsorption, vapour deposition, spray drying and coating.

With the process of the invention, catalysts and catalyst carriers according to the invention can be prepared. Hence, with the process of the invention a catalyst or a catalyst carrier can be prepared which comprises:
35 to 99.9 wt %, preferably 45 to 99.9 wt %, more preferably 75 to 99.9 wt %, of metal oxide and
0.1 to 50 wt % of silanized silica particles,
   calculated on the total weight of the catalyst or catalyst carrier.

Also all embodiments of the catalysts and catalyst carriers according to the invention which are listed above can be prepared with the process of the invention.

Process Step (a)

In step (a) of the process of the invention, the following are contacted:
metal oxide, and
silanized silica, suitably silanized silica particles, more suitably a dispersion of silanized silica particles, even more suitably a dispersion of silanized colloidal silica particles, and
a solvent and/or one or more shaping aids.

In the present specification, by “dispersion of silanized colloidal silica particles” reference is made to a mixture wherein insoluble silanized silica particles are suspended throughout another substance (for example water). In particular, for such dispersion to be formed, it may be required that the average particle diameter is not too large. Suitably, in the present invention, the silanized silica particles may have an average particle diameter of 1 to 1,000 nanometers (nm), preferably 2 to 100 nm, more preferably 2 to 40 nm, most preferably 2 to 10 nm.

As metal oxide may be used one or more of the metal oxides described above for the catalyst and catalyst carrier of the invention.

As silanized silica and silanized silica particles may be used silanized silica and silanized silica particles as described above for the catalyst and catalyst carrier of the invention.

In case a shaping aid is used which comprises a solvent, it may not be necessary to add additional solvent.

The dispersion of silanized silica particles that may be used in the process of the present invention preferably is an aqueous dispersion. Additionally or alternatively, water may be used as solvent. Additionally or alternatively, a shaping aid comprising water may be used.

Preferably a shaping aid is used in the process of the invention. More preferably the shaping aid comprises one or more of: cellulose, polycellulose, cellulose ether, polyethylene oxide, and polyvinyl alcohol.

A suitable polycellulose is polycellulose as obtainable from DOW, e.g. Walocel. A suitable cellulose ether is cellulose ether as obtainable from DOW, e.g. Methocel. A suitable polyethylene oxide is polyethylene as obtainable from Dow, for example Polyox. A suitable polyvinyl alcohol is polyvinyl alcohol as obtainable from Sigma-Aldrich, e.g. Mowiol.

More preferably polyethylene oxide is used as shaping aid. Even more preferably polyethylene oxide is used as shaping aid, and additionally another shaping aid is used.

Still more preferably polyethylene oxide is used as shaping aid, and additionally polycellulose, cellulose ether, and/or polyvinyl alcohol is/are used as shaping aid(s). For example, in the process of the invention Polyox and Walocel may be used, or Polyox and Methocel, or Polyox and Mowiol.

Depending on the metal oxide, the shaping aid(s) can be chosen.

Process Step (b)

In step (b) of the process of the invention, the material obtained in step (a) is shaped (or formed). Shaping (or forming) may be performed by means of spray drying, pelletizing, (wheel) pressing, extrusion, or application on a metal support (like a metal wire or a metal flake), preferably by means of extrusion. In the present specification, “extrusion” means a process used to create objects of a fixed cross-sectional profile, wherein a material is pushed through a die of the desired cross-section. Extrusion is different from for example forming layers through deposition of a sol onto a support which deposition may be followed by evaporation of solvent.

Process Step (c)

In step (c) of the process of the invention, the material obtained in step (b) is dried and/or heated, suitably heated, at a temperature in the range of from 60 to 700° C., preferably 60 to 600° C., more preferably 60 to 450° C. Said heating may take place in several steps at different temperatures. In a first step, heating may take place at a relatively low temperature, for example of from 60 to 200° C., at which temperature drying may be effected, followed by heating in a further step at a relatively high temperature, for example of from 200 to 700° C., suitably 300 to 600° C.

Depending on the metal oxide, the temperature can be chosen in step (c). Also the atmosphere in which step (c) is performed can be chosen depending on the metal oxide. Step (c) is preferably performed in air.

Use in Catalytic Reaction

The present invention also relates to the use of a catalyst according to the invention or a catalyst prepared by the process according to the invention in a catalytic reaction. The present invention also relates to the use of a catalyst comprising a catalyst carrier according to the invention or a catalyst comprising a catalyst carrier prepared by the process according to the invention in a catalytic reaction.

Use in Alkane Oxidative Dehydrogenation

In particular, the present invention also relates to the use of a catalyst according to the invention, or a catalyst prepared by the process according to the invention, a catalyst comprising a catalyst carrier according to the invention or a catalyst comprising a catalyst carrier prepared by the process according to the invention in an alkane oxidative dehydrogenation (alkane ODH) and/or alkene oxidation reaction. Accordingly, the present invention also relates to a process of the oxidative dehydrogenation of an alkane containing 2 to 6 carbon atoms and/or the oxidation of an alkene containing 2 to 6 carbon atoms, comprising contacting oxygen and the alkane containing 2 to 6 carbon atoms and/or the alkene containing 2 to 6 carbon atoms with a catalyst according to the invention, or a catalyst prepared by the process according to the invention, a catalyst comprising a catalyst carrier according to the invention or a catalyst comprising a catalyst carrier prepared by the process according to the invention.

In the alkane oxidative dehydrogenation process and/or alkene oxidation process of the present invention, 1) oxygen (O₂) and 2) an alkane containing 2 to 6 carbon atoms and/or alkene containing 2 to 6 carbon atoms may be fed to a reactor. Said components may be fed to the reactor together or separately. That is to say, one or more feed streams, suitably gas streams, comprising one or more of said 2 components may be fed to the reactor. For example, one feed stream comprising oxygen and the alkane and/or alkene may be fed to the reactor. Alternatively, two or more feed streams, suitably gas streams, may be fed to the reactor, which feed streams may form a combined stream inside the reactor. For example, one feed stream comprising oxygen and another feed stream comprising the alkane and/or alkene may be fed to the reactor separately.

In the alkane oxidative dehydrogenation process and/or alkene oxidation process of the present invention, oxygen and the alkane containing 2 to 6 carbon atoms and/or alkene containing 2 to 6 carbon atoms are suitably fed to the reactor in the gas phase.

Preferably, in the present alkane oxidative dehydrogenation process and/or alkene oxidation process, that is to say during contacting the oxygen and the alkane and/or alkene with the catalyst, the temperature is of from 300 to 500° C. More preferably, said temperature is of from 310 to 450° C., more preferably of from 320 to 420° C., most preferably of from 330 to 420° C.

Still further, in the present alkane oxidative dehydrogenation process and/or alkene oxidation process, that is to say during contacting the oxygen and the alkane and/or alkene with the catalyst, typical pressures are 0.1-30 or 0.1-20 bara (i.e. “bar absolute”). Further, preferably, said pressure is of from 0.1 to 15 bara, more preferably of from 1 to 8 bara, most preferably of from 3 to 8 bara.

Preferably, in the alkane oxidative dehydrogenation process of the present invention, the alkane containing 2 to 6 carbon atoms is a linear alkane in which case said alkane may be selected from the group consisting of ethane, propane, butane, pentane and hexane. Further, preferably, said alkane contains 2 to 4 carbon atoms and is selected from the group consisting of ethane, propane and butane. More preferably, said alkane is ethane or propane. Most preferably, said alkane is ethane.

Further, preferably, in the alkene oxidation process of the present invention, the alkene containing 2 to 6 carbon atoms is a linear alkene in which case said alkene may be selected from the group consisting of ethylene, propylene, butene, pentene and hexene. Further, preferably, said alkene contains 2 to 4 carbon atoms and is selected from the group consisting of ethylene, propylene and butene. More preferably, said alkene is ethylene or propylene.

The product of said alkane oxidative dehydrogenation process may comprise the dehydrogenated equivalent of the alkane, that is to say the corresponding alkene. For example, in the case of ethane such product may comprise ethylene, in the case of propane such product may comprise propylene, and so on. Such dehydrogenated equivalent of the alkane is initially formed in said alkane oxidative dehydrogenation process. However, in said same process, said dehydrogenated equivalent may be further oxidized under the same conditions into the corresponding carboxylic acid which may or may not contain one or more unsaturated double carbon-carbon bonds. As mentioned above, it is preferred that the alkane containing 2 to 6 carbon atoms is ethane or propane. In the case of ethane, the product of said alkane oxidative dehydrogenation process may comprise ethylene and/or acetic acid, preferably ethylene. Further, in the case of propane, the product of said alkane oxidative dehydrogenation process may comprise propylene and/or acrylic acid, preferably acrylic acid.

The product of said alkene oxidation process comprises the oxidized equivalent of the alkene. Preferably, said oxidized equivalent of the alkene is the corresponding carboxylic acid. Said carboxylic acid may or may not contain one or more unsaturated double carbon-carbon bonds. As mentioned above, it is preferred that the alkene containing 2 to 6 carbon atoms is ethylene or propylene. In the case of ethylene, the product of said alkene oxidation process may comprise acetic acid. Further, in the case of propylene, the product of said alkene oxidation process may comprise acrylic acid.

In addition to oxygen and the alkane and/or alkene, an inert gas may also be fed. Said inert gas may be selected from the group consisting of the noble gases and nitrogen (N₂). Preferably, the inert gas is nitrogen or argon, more preferably nitrogen. Said oxygen is an oxidizing agent, thereby resulting in oxidative dehydrogenation of the alkane and/or oxidation of the alkene. Said oxygen may originate from any source, such as for example air. Ranges for the molar ratio of oxygen to the alkane and/or alkene which are suitable, are of from 0.01 to 1, more suitably 0.05 to 0.5. Said ratio of oxygen to the alkane and/or alkene is the ratio before oxygen and the alkane and/or alkene are contacted with the catalyst. In other words, said ratio of oxygen to the alkane and/or alkene is the ratio of oxygen as fed to the alkane and/or alkene as fed. Obviously, after contact with the catalyst, at least part of the oxygen and alkane and/or alkene gets consumed.

Examples of oxydehydrogenation processes, including process conditions, are for example disclosed in above-mentioned U.S. Pat. No. 7,091,377, WO2003064035, US20040147393, WO2010096909 and US20100256432, the disclosures of which are herein incorporated by reference.

The amount of the catalyst in said process is not essential. Preferably, a catalytically effective amount of the catalyst is used, that is to say an amount sufficient to promote the alkane oxydehydrogenation and/or alkene oxidation reaction.

In general, water is formed during the alkane ODH and/or alkene oxidation reaction(s) that take place in said process, which water may end up in the product stream in addition to the desired product. Water may easily be separated from said product stream, for example by cooling down the product stream from the reaction temperature to a lower temperature, for example room temperature, so that the water condenses and can then be separated from the product stream.

### EXAMPLES

Catalysts and catalyst carriers according to the invention were prepared with the process of the invention. Comparative catalysts and catalyst carriers were prepared by changing the ingredients.

### Example 1

A catalyst carrier extrudate was prepared. 6.6 grams of ZSM-5 (zeolite) powder was mixed with 3.0 grams of Sipernat 50 (SiO₂) powder and 0.1 gram of Polyox WSR301 (a shaping aid) in a mixer at 2500 rpm for 30 seconds. The resulting mixture was transferred into a kneader and during mixing/kneading, a mixture comprising 1) 2.32 grams of a solution of 0.6 wt. % Walocel (a shaping aid) in water and 2) 4.4 grams of Bindzil CC301 was added incrementally till the mixture became an extrudable paste. Bindzil CC301 is an aqueous dispersion comprising 30 wt. % of silanized colloidal silica particles having an average particle diameter of about 7 nm. The resulting paste was extruded by means of an extruder into cylinder shaped bodies. The resulting extrudate was air dried at 80° C. for 2 hours and then calcined in static air at 575° C. for 1 hour. The resulting catalyst carrier extrudate comprised about 60 wt. % of zeolite (ZSM-5), about 28 wt. % of non-silanized silica (Sipernat 50) and about 12 wt. % of silanized silica (Bindzil). The preparation and the testing of the catalyst carrier extrudate was repeated twice.

### Comparative Example 1

A catalyst carrier extrudate was prepared according to Example 1, but instead of Bindzil CC301 (silanized silica), Bindzil 30NH3/220 (also ex AkzoNobel) was used. Bindzil 30NH3/220 is an aqueous NH₃ stabilized dispersion comprising 30 wt. % of non-silanized colloidal silica particles having an average particle diameter of about 15 nm. The resulting catalyst carrier extrudate comprised about 60 wt. % of zeolite (ZSM-5), about 28 wt. % of non-silanized silica (Sipernat 50) and about 12 wt. % of non-silanized silica (Bindzil).

Results Strength Measurements

Flat Plate Crushing Strength (FPCS) measurements at 371° C. were performed on the catalyst carrier extrudates of Example 1 and Comparative Example 1. The catalyst carrier extrudate of Example 1 was about 2 times stronger than the catalyst carrier extrudate of Comparative Example 1. FPCS-371 Example 1=191 N/cm; FPCS-371 Comparative Example 1=101 N/cm.

Preparation of Catalyst Powder A

A mixed metal oxide catalyst powder containing molybdenum (Mo), vanadium (V), niobium (Nb) and tellurium (Te) was prepared, for which catalyst the molar ratio of said 4 metals was Mo₁V_{0.29}Nb_{0.17}Te_{0.12}.

Two solutions were prepared. Solution 1 was obtained by dissolving 15.8 g of ammonium niobate oxalate and 4.0 g of anhydrous oxalic acid dihydrate in 160 ml of water at room temperature. Solution 2 was prepared by dissolving 35.6 g of ammonium heptamolybdate, 6.9 g of ammonium metavanadate and 5.8 g of telluric acid (Te(OH)₆) in 200 ml of water at 70° C. 7.0 g of concentrated nitric acid was then added to solution 2. The 2 solutions were combined which yielded an orange gel-like precipitate. The mixture was spray dried with the aid of a Buchi-290 spray drier.

The dried material was further dried in static air at 120° C. for 16 hours, milled to a fine powder and then calcined in static air at a temperature of 325° C. for 2 hours. After the air calcination, the material was further calcined in a nitrogen (N₂) stream at 600° C. for 2 hours. The resulting catalyst powder A was a powder comprising the mixed metal oxide in the orthorhombic M1 crystalline phase.

### Example 2

Catalyst extrudates were prepared. 10 grams of catalyst powder A as prepared in the above way was mixed with 3.12 grams of Sipernat 500 LS (SiO₂) powder and 0.13 gram of Polyox WSR301 (a shaping aid) in a mixer at 2500 rpm for 30 seconds. The resulting mixture was transferred into a kneader and during mixing/kneading, a mixture comprising 1) 4.8 grams of a solution of 0.6 wt. % Walocel (a shaping aid) in water and 2) Bindzil was added incrementally till the mixture became an extrudable paste. The amount of Bindzil added can be derived from the data in the Table below. Said Bindzil was either Bindzil CC151 HS (“type I” in the Table below) or Bindzil CC301 (“type II” in the Table below). Bindzil CC301 is an aqueous dispersion comprising 30 wt. % of silanized colloidal silica particles having an average particle diameter of about 7 nm. Bindzil CC151 HS is an aqueous dispersion comprising 15 wt. % of silanized colloidal silica particles having an average particle diameter of about 5 nm. The resulting paste was extruded by means of an extruder into cylinder shaped bodies. The resulting extrudate was air dried at 80° C. for 2 hours and then calcined in static air at 325° C. for 2 hours. The composition of the resulting catalyst extrudates is shown in the Table below.

### Comparative Example 2

A catalyst extrudate was prepared according to Example 2, but no Bindzil was used. Instead of the mixture comprising Walocel and Bindzil, 6.5 grams of a solution of 0.6 wt. % Walocel in water was used. The resulting catalyst extrudate comprised about 76 wt. % of the mixed metal oxide (catalyst powder A) and about 24 wt. % of non-silanized silica (Sipernat 500 LS).

Results Strength Measurements

Flat Plate Crushing Strength (FPCS) measurements were performed on the catalyst extrudates of Example 2 and Comparative Example 2. The results are summarized in the Table below.

Experiment

Silanized silica
(wt. % in final
catalyst)⁽¹⁾

| | Bindzil | Bindzil | FPCS-25 | FPCS-325 |
|---|---|---|---|---|
| | type I | type II | (N/cm)⁽²⁾ | (N/cm)⁽²⁾ |
| Comp Ex 2 | 0 | 0 | 29 | 48 |
| Ex 2A | 1 | 0 | 52 | 81 |
| Ex 2B | 3 | 0 | 86 | 152 |
| Ex 2C | 5 | 0 | 65/60 | |
| Ex 2D | 0 | 1 | 48 | 85 |
| Ex 2E | 0 | 3 | 65/71 | |
| Ex 2F | 0 | 5 | 47 | 98 |
| Ex 2G | 0 | 10 | 66 | 114 |

⁽¹⁾In addition to silanized silica, the catalyst comprised the mixed metal oxide (catalyst powder A) and non-silanized silica (Sipernat 500 LS) in a weight ratio of about 10:3.
⁽²⁾FPCS-25 stands for a Flat Plate Crushing Strength measurement at 25° C., FPCS-325 was measured at 325° C.

The results clearly show that the strength of the catalyst extrudates of Examples 2A to 2G, which are according to the invention, is higher than the strength of the catalyst extrudate of Comparative Example 2, both for FPCS-25 and for FPCS-325.

### Example 3: Catalyst Testing in Oxidative Dehydrogenation (ODH)

A catalyst extrudate was prepared in the same way as in Example 2, with the exception that in the extrusion step trilobe shaped bodies were formed instead of cylinder shaped bodies.

In order to test the above-mentioned catalyst extrudate in the oxidative dehydrogenation (ODH) of ethane in a small laboratory setup, it had to be crushed. In case a crushed catalyst extrudate shows catalytic activity, the same is valid for the uncrushed catalyst extrudate. The crushed material was sieved using a sieve having a mesh size of 40-80 mesh. The sieved material having a size of 40-80 mesh was then used in the following ethane ODH experiment.

The ethane ODH experiment was performed within a small-scale testing unit comprising a vertically oriented, cylindrical, quartz reactor having an inner diameter of 2.0 mm. The catalyst was loaded in the reactor. The catalyst bed height was about 6 cm. On top of the catalyst bed, another bed having a height of 8 cm was placed which latter bed contained inert silicon carbide (SiC) particles having a particle size of 0.8 mm. In this experiment, a gas stream comprising 63 vol. % of ethane, 21 vol. % of oxygen (O₂) and 16 vol. % of nitrogen (N₂) was fed to the top of the reactor and then sent downwardly through the catalyst bed to the bottom of the reactor. Said gas stream was a combined gas stream comprising a flow of ethane having a rate of 3.00 Nl/hour, a flow of oxygen having a rate of 1.00 Nl/hour and a flow of nitrogen having a rate of 0.77 Nl/hour. “Nl” stands for “normal litre” as measured at standard temperature and pressure, namely 32° F. (0° C.) and 1 bara (100 kPa). The gas hourly space velocity was set to about 4,000 Nl/liter catalyst/hour. The pressure in the reactor was 4.7 bara. The reactor was heated such that the catalyst temperature was about 300° C. This condition was maintained for a number of hours.

Following this initial period at the initial temperature of about 300° C., the temperature was increased stepwise up to about 340° C. Further, at each temperature, the conversion was monitored for a number of hours. The conversion of ethane and the product composition were measured with a gas chromatograph (GC) equipped with a thermal conductivity detector (TCD) and with another GC equipped with a flame ionization detector. Acetic acid by-product and water from the reaction were trapped in a quench pot.

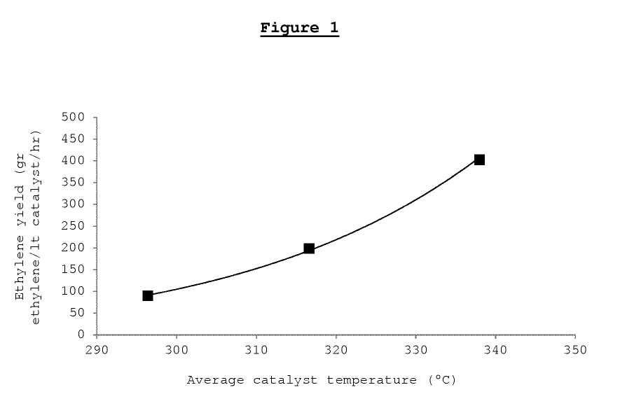

FIG. 1 shows the catalyst performance data in ethane ODH. In FIG. 1, the catalyst productivity as a function of catalyst temperature is shown. By said catalyst productivity, reference is made to space-time yield which was measured as grams of ethylene produced per liter of catalyst per hour. Further, by said catalyst temperature, reference is made to the average of the top catalyst temperature and the bottom catalyst temperature, wherein the top catalyst temperature is the temperature measured in the catalyst bed at a position which is about 0.5 cm from the top and the bottom catalyst temperature is the temperature measured in the catalyst bed at a position which is about 0.5 cm from the bottom.

It appears from the data in FIG. 1 that the catalyst according to the invention prepared by a process according to the invention can advantageously be used in converting ethane into ethylene by oxidative dehydrogenation (ethane ODH), in a relatively low temperature range of 290 to 340° C.

1. A catalyst or a catalyst carrier comprising:
35 to 99.9 wt % of metal oxide and
0.1 to 50 wt % of silanized silica particles,
calculated on the total weight of the catalyst or catalyst carrier, wherein the amount of metal oxide does not include the amount of silanized silica particles.

2. The catalyst or a catalyst carrier according to claim 1, which is an extrudate.

3. The catalyst or a catalyst carrier according to claim 1, wherein the silanized silica particles have an average particle diameter of 1 to 1,000 nm.

4. The catalyst or a catalyst carrier according to claim 1, which comprises:
silanized silica particles, and
silica and/or cerium oxide, and
one or more of the following:
molybdenum oxide, molybdenum-vanadium oxide, molybdenum-vanadium-niobium oxide, molybdenum-vanadium-niobium-tellurium oxide, molybdenum-vanadium-niobium-tellurium-antimony oxide, molybdenum-vanadium-antimony oxide, titania, silica alumina and zeolite.

5. A process for the preparation of a catalyst or catalyst carrier, comprising the steps of:
(a) contacting
metal oxide,
silanized silica, suitably silanized silica particles, more suitably a dispersion of silanized silica particles, even more suitably a dispersion of silanized colloidal silica particles,
a solvent and/or one or more shaping aids;
(b) shaping or forming, suitably shaping, the material obtained in step (a);
(c) drying and/or heating, suitably heating, the material obtained in step (b):
at a temperature in a range of from 60 to 700° C.

6. The process according to claim 5, wherein step (b) is performed by means of extrusion.

7. The process according to claim 5, wherein the dispersion of silanized silica particles is an aqueous dispersion and/or water is used as solvent and/or wherein a shaping aid comprising water is used.

8. Use of a catalyst according to claim 1 in a catalytic reaction.

9. A process for the oxidative dehydrogenation of an alkane containing 2 to 6 carbon atoms and/or the oxidation of an alkene containing 2 to 6 carbon atoms, comprising contacting oxygen and the alkane containing 2 to 6 carbon atoms and/or the alkene containing 2 to 6 carbon atoms with a catalyst according to claim 1.

10. Use of a catalyst prepared by the process according to claim 5 in a catalytic reaction.

11. Use of a catalyst comprising a catalyst carrier according to claim 1 in a catalytic reaction.

12. Use of a catalyst comprising a catalyst carrier prepared by the process according to claim 5 in a catalytic reaction.

13. A process for the oxidative dehydrogenation of an alkane containing 2 to 6 carbon atoms and/or the oxidation of an alkene containing 2 to 6 carbon atoms, comprising contacting oxygen and the alkane containing 2 to 6 carbon atoms and/or the alkene containing 2 to 6 carbon atoms with a catalyst prepared by the process according to claim 5.

14. A process for the oxidative dehydrogenation of an alkane containing 2 to 6 carbon atoms and/or the oxidation of an alkene containing 2 to 6 carbon atoms, comprising contacting oxygen and the alkane containing 2 to 6 carbon atoms and/or the alkene containing 2 to 6 carbon atoms with a catalyst comprising a catalyst carrier according to claim 1.

15. A process for the oxidative dehydrogenation of an alkane containing 2 to 6 carbon atoms and/or the oxidation of an alkene containing 2 to 6 carbon atoms, comprising contacting oxygen and the alkane containing 2 to 6 carbon atoms and/or the alkene containing 2 to 6 carbon atoms with a catalyst comprising a catalyst carrier prepared by the process according to claim 5.
